(19) United States
(12) Patent Application Publication
(10) Pub. No.: US 2005/0059639 A1
(43) Pub. Date: Mar. 17, 2005

(54) Ophthalmic compositions and method for treating eye discomfort and pain

(76) Inventor: Edward T. Wei, Berkeley, CA (US)

Correspondence Address: Edward T. Wei, 480 Grizzly Peak Blvd, Berkeley, CA, 94708, US

(21) Appl. No.: 10/660,905

(22) Filed: Sep. 11, 2003

(51) Int. Cl. A61K 31/66

(52) U.S. Cl. 514/142

(57) ABSTRACT

Eye discomfort is reduced by administering drops of an inventive composition containing a trialkyl phosphine oxide in an ophthalmic solution. The preferred method of administration is to drip the solution onto the medial canthus of the closed eye and to keep the eye closed until at least one minute after instillation. The preferred trialkyl phosphine oxide is selected for potency, long duration of action, and the absence of irritancy. A hydrocarbon polyol or a similar demulcent may be added to the composition in order to further reduce irritancy. The concentration of the trialkyl phosphine oxide in the ophthalmic solution is preferably in an amount of at least about 0.001 wt. % to about 0.5% (10 μg/ml to 5 mg/ml) of the composition.

### BACKGROUND OF THE INVENTION

1. Field of the Invention

This invention generally relates to ophthalmic compositions, and more particularly to compositions that contain a trialkyl phosphine oxide and are useful to treat the eyes for eye irritation, eye itching, and eye pain, for example to reduce post-operative eye pain in a patient following eye surgery.

2. Description of Related Art

The eye surfaces are exposed to the external environment. These anatomical structures—eyelids, front part of the eyeball, conjunctiva, lachrymal system, precorneal film and cornea—are subject to injury by physical, chemical and biological agents. The characteristic symptoms of injury to the eye are impeded vision, discomfort, itching, irritation, stinging and burning sensations, and pain. The signs of injury in the vascularized portions of the eye are redness, swelling and increased blood flow. Ophthalmic products such as solutions, ointments, and inserts are used to manage the causes and the signs and symptoms of eye injury.

To medicate the eye, ophthalmic eye drops, as a drug delivery system, are preferred to ointments and inserts because of ease and costs of preparation, patient familiarity with procedures of drug administration, and the lower frequency of side effects. An ideal active ingredient in ophthalmic solutions should be soluble and miscible in aqueous media at normal ocular pH and tonicity. Moreover, the drug should be stable, non-toxic, long acting, and sufficiently potent to counteract dilution of drug concentration by blinking and tearing.

Phosphine oxides having a physiological cooling effect were described by Rowsell and Spring in U.S. Pat. No. 4,070,496, issued Jan. 24, 1978. Watson et al also mentioned these compounds briefly in a publication entitled New Compounds with the Menthol Cooling Effect. J. Soc. Cosmet. Chem. 29: 185-200, 1978. The principal intended use of these compounds was as additives to toiletries, cosmetics, and comestibles.

Rowsell and Spring in Example 3 of their U.S. Pat. No. 4,070,496 described an eye lotion that contained 12.95% Witch Hazel (an astringent) and di-isopentyl-sec-butyl phosphine oxide at 0.005%. Eye lotions are intended for washing, bathing or flushing the eye, often contain an astringent, and are used for first aid or similar emergency purposes. Astringents are locally acting pharmacologic agents, which, by precipitating protein, help to clear mucus from the outer surface of the eye. (Section 21 Code of Federal Regulations, Part 349). Eye lotions are packaged in relatively large volume containers (100 ml or greater) with an eye-cup, and, if no bactericide is included, the lotion is to be discarded 24 hours after opening the container. In using eye lotions, patients are advised to fill a sterilized eye-cup half-full with lotion, press the cup to the eye, tilt the head backwards, open the eyelids wide and rotate the eyeball for a thorough rinse.

Eye lotions, used to rinse and clean, do not address eye pain where ophthalmic delivery of a long acting and sufficiently potent drug is needed to alleviate pain. There is a need for eye drop compositions providing relief of eye pain, discomfort, itch, or irritation due to allergy, and eye disease such as conjunctivitis, and especially following eye surgery, such as refractive eye surgery (e.g. LASIK and PRK) and cataract surgery.

Compositions capable of suppressing eye irritation, itch and especially pain are the subject of this invention.

### BRIEF SUMMARY OF THE INVENTION

In one aspect of the present invention, an eye drop composition is provided that is useful to reduce eye discomfort and comprises one or more doses of an ophthalmic solution having therein a pharmaceutically effective amount of a trialkyl phosphine oxide of Formula 1
(R₁R₂R₃)P═O Formula 1

wherein R₁ is an alkyl radical containing at least 3 carbon atoms, R₂ is an alkyl radical containing at least 3 carbon atoms or a cycloalkyl radical, R₃ is an alkyl radical, and R₁, R₂ and R₃ total of from 13-17 carbon atoms, and wherein the pharmaceutically effective amount is at least about 0.0001 to 0.1 weight percent (0.1 μg/ml to 1 mg/ml) of the composition. The one or more doses are adapted for therapeutic efficacy in treating eye discomfort by including one or more of:
a.) a selection of R₁ as n-C₅H₁₁, n-C₆H₁₃, n-C₇H₁₅ or n-C₈H₁₇, R_{as iso-C}₃H₇, sec-C₄H₉, tert-C₄H₉ or iso-C₅H₁₁ and R₃ as n-C₃H₇, iso-C₃H₇, sec-C₄H₉, or n-C₄H₉;
b.) an adjunct to reduce irritancy from the trialkyl phosphine oxide; and
c.) instructions to the user for applying the solution indirectly to the eye.

Preferred in eye drop compositions are buffered, isotonic and substantially non-astringent solutions.

A particularly preferred composition useful for treating eye pain is a buffered, isotonic, non-astringent ophthalmic solution is wherein R₁ R₁ is n-C₅H₁₁, n-C₆H₁₃, n-C₇H₁₅ or n-C₈H₁₇, R₂ is iso-C₃H₇, sec-C₄H₉, tert-C₄H₉ iso-C₅H₁₁ and R₃ is n-C₃H₇, iso-C₃H₇, sec-C₄H₉, or n-C₄H₉.

In one aspect of practicing the invention, a therapeutically effective amount of a trialkyl phosphine oxide solution for relief of eye pain, discomfort, itch, or irritation is administered to or by a patient where a unit dose is applied onto the nasal corner (medial canthus) of a closed eye and the eye is kept closed for at least about one minute.

More preferably a method of reducing eye discomfort in a user is wherein a buffered, isotonic ophthalmic solution is provided having therein a pharmaceutically effective amount of a trialkyl phosphine oxide of Formula 1
(R₁R₂R₃)P═O Formula 1

wherein R₁ is an alkyl radical containing at least 3 carbon atoms, R₂ is an alkyl radical containing at least 3 carbon atoms or a cycloalkyl radical, R₃ is an alkyl radical, and R₁, R₂ and R₃ total of from 13-17 carbon atoms, wherein the solution is either provided as a unit dose or is determinable as a unit dose, and the solution user to instructed to administer the unit dose onto the nasal corner (medial canthus) of a closed eye and to keep the eye closed for at least one minute.

Trialklyl phosphine oxide compositions formulated as an eye drops solution and administered in accordance with this invention provide a non-painful, non-inflammatory, cooling effect that typically lasts for up to two (2) hours and greater. Compositions of the present invention are useful in treating ocular irritation, itching, and pain associated with various physical, chemical, and biological agents. For example, compositions of the present invention are highly effective in relieving eyelid and conjunctival irritation and itch from seasonal allergens and inflammation, pain and itch from ocular viral infections, pain and irritation from contact lens use, and pain and irritation from eye surgery (see Examples, infra). Furthermore, compositions of the present invention exert their beneficial effects when administered directly onto the eye and also when applied to the immediately adjacent anatomic features (e.g., eyelids, eye socket, and bridge of the nose).

Other advantages and aspects of the present invention will be understood by reading the following detailed description and the accompanying claims.

### DETAILED DESCRIPTION OF THE INVENTION

Trialkyl phosphine oxides produce significant stinging and burning sensations at concentrations above about 0.005 weight percent when applied directly to the eye when the eyelids are open. However, I have discovered that trialkyl phosphine oxide compositions can be formulated into eye drops solution and administered for a therapeutic effect that typically lasts for up to two (2) hours or more, even when formulated at concentrations on the order of about 0.2 weight percent.

Compositions of the present invention are useful in treating ocular irritation, itching, and pain associated with various physical, chemical, and biological agents. For example, I have found that the inventive compositions are highly effective in relieving irritation and itch from seasonal allergens and inflammation, pain and itch from ocular viral infections, pain and irritation from contact lens use, and pain from eye surgery (see Examples, infra). Furthermore, I have discovered that compositions of the present invention exert their beneficial effects when administered onto the margins of the eyelids and eye surface, and also when applied to the immediately adjacent anatomic features (e.g., eyelids, eye socket, and bridge of the nose).

These and other aspects of the invention will be more fully described and exemplified hereinafter.

Definitions:

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The terms treatment”, “therapy,” “beneficial effect,” and “therapeutic effect” have equivalent meanings in the context of the present invention and indicate a positive change or changes in a subject's status. The change(s) can be either subjective or objective and can relate to features such as symptoms or signs of a disease, disorder, or condition being treated. For example, if the subject notes decreased eye fatigue and strain, decreased itching, reduced discomfort, or decreased pain, then successful treatment has occurred. Similarly, if the clinician notes objective changes, such as decreased redness and swelling, then treatment has also been successful.

The terms “drug”, “pharmacological agent”, “pharmaceutical agent”, “active agent”, and “agent” are herein used interchangeably and are intended to have their broadest meaning as to any therapeutically-active substance which is delivered to a living organism to produce a desired, usually beneficial effect.

The term “pharmaceutically-acceptable” refers to a substance which does not interfere with the effectiveness or the biological activity of the active ingredients and which is not toxic to a subject to which it is administered.

The term “therapeutically-effective amount” is used herein to denote any amount of the formulation which causes a substantial improvement in a disease, disorder, or condition when applied to the affected areas. The amount will vary with the condition being treated, the stage of advancement of the condition, and the type and concentration of formulation applied. Appropriate amounts in any given instance will be readily apparent to those skilled in the art or capable of determination by routine experimentation.

“Disease” as used herein is defined as a physiological condition that has developed in response to an exogenous and/or endogenous stimulus or stimuli in which the organism is adversely affected in some manner. The adverse effect can comprise minor irritation, minor itching, and minor pain up to major irritation, major itching, and major pain. The adverse effect can result from exposure to a physical, chemical, and/or biological agent. The disease may elicit clinical signs such as vasodilation in the capillaries of the eye (e.g., conjunctivitis) or may be limited to symptoms as described by the subject. A disease can also be a disorder but, as used herein, the term “disorder” is broader than disease, as it encompasses conditions that are not characterized as a disease or diseases by medical practitioners. For example, minor discomfort (e.g., eye strain and fatigue, minor irritation, minor itching) felt in the eye by a subject, e.g., from the use of contact lenses, may not be diagnosed by an ophthalmologist or optometrist as a disease per se but is nevertheless a disorder as contemplated herein. “Buffering agent” refers to a substance which stabilizes the pH of solutions against changes produced by introduction of acids or bases from such sources as drugs, body fluids, tears, etc.

“Demulcent” typically refers to an agent, usually a water-soluble polymer, which is applied topically to the eye to protect and lubricate mucous membrane surfaces and to relieve dryness and irritation. The types of demulcent polymers permitted by the Federal Food and Drug Administration in ophthalmic solutions are defined concentrations of cellulose derivatives (0.2-2.5%, methylcellulose, hydroxyethylcellulose, hydroxypropylcellulose and carboxymethylcellulose), dextran 70 (0.1%), gelatin (0.01%), polyols (0.2 to 1%): glycerin, polyethylene glycol 300, polyethylene glycol 400, polysorbate 80, propylene glycol, polyvinyl alcohol (to 4%) and povidone (polyvinylpyrrolidone, to 2%). However, as will be further discussed hereinafter, I have discovered that certain demulcents surprisingly function in combination with trialkyl phosphine oxides to reduce or substantially eliminate the sting associated with higher concentrations of trialkyl phosphine oxides in ophthalmic compositions of the invention.

“Isotonicity” refers to a state or quality in which the osmotic pressure in two fluids is equal.

A “symptom” or “symptoms” of eye injury refer the subjective sensations of the individual when tissues of the eye are irritated. The descriptive terms used would include words such as: “it smarts,” “it stings,” “it itches,” “it burns,” “it aches,” “it hurts” or just simply “ouch.”

“Subject” as used herein includes experimental animals such as mice, rats, dogs, rabbits, and non-human primates. When a subject is an animal or human undergoing medical diagnosis and/or treatment by a medical practitioner, then the animal or human is also referred to as a “patient.” A human involved in clinical trials is also referred to as a patient.

Anatomical Features of the Eye:

The anterior segment of the eye consists of the eyelids and the front part of the eyeball. Within these structures are:
the nasolachrymal system which secretes, distributes and excretes tears;
the precorneal tear film, a fluid layer that covers the corneal epithelium, conjunctiva and the walls of the conjunctival cul-de-sac. The tear volume is about 7 μL, the volume of the cul-de-sac is about 30 μL, and commercial eye drops are 50 to 75 μL per drop. Tears nourish the cornea, flush and dilute unwanted materials from the orbit, and provide lubrication for movement of eyelids and eyeball;
the conjunctiva, a thin, vascularised mucous membrane that lines the posterior surface of the eyelids and outer regions of the cornea; and
the cornea, an optically transparent tissue that centrally covers the pupil. The corneal epithelium is about 5 to 6 layers of cells, contains sensory nerve endings, but does not contain blood vessels. The turnover rate of corneal epithelium is about one layer of cells per day.

Ophthalmic Diseases:
Ophthalmic diseases contemplated for treatment by the compositions and methods of the present invention, preferably by topical application of trialkyl phosphine oxide solutions formulated as eye drops include, but are not limited to:
blepharitis or inflammation of the eyelids,
dry eye syndrome (keratoconjunctivitis sicca), the inadequate wetting of the ocular surface caused, for example, by inadequate tear secretion or rapid evaporation of tears because of poor tear quality,
conjunctivitis, an inflammation of the conjunctiva that is most commonly caused by allergens, smoke, and pollutants, but may also be caused by bacterial and viral infection, and physical agents such as trauma, wind and sunlight,
keratitis, an inflammation of the cornea, that may be caused by physical trauma, such as cataract surgery or refractive eye surgery, and also by bacterial or viral infection. A corneal abrasion is an injury to the epithelium that is superficial enough not to involve the basement membrane. It occurs mainly after mechanical trauma. A corneal ulcer is a defect that involves the stroma, past Bowman's membrane; the lesion can easily become infected and lead to loss of vision.
iritis (anterior uveitis), an inflammation of the iris, a condition that is rare but associated with considerable pain and inflammation
general eye discomfort, for example, caused by extended wear of contact lenses, by eye strain, by excessive exposure to the sun.

Other pharmacological agents used in eye drops for the treatment of the above as well as other ophthalmic diseases and disorders include, but are not limited to, antibiotics such as chloramphenicol, gentamicin; antiviral agents such as cidofovir, valaciclovir, trifluridine; antihistamines and mast-cell stabilizers such as olopatadine (Patanol®) and nedocromil; anti-inflammatory steroids such as betamethasone and prednisolone; non-steroidal anti-inflammatory agents such as ketorolac and diclofenac; immunomodulators such as interferons; and other drugs such as miotics, sympathomimetics, β-adrenergic receptor blockers, and local anesthetics.

Ophthalmic diseases and disorders cause considerable discomfort as well as anxiety in patients, in part because of fear of loss of visual function. A common pathway for expression of tissue damage is the sensations that accompany injury. Symptoms of discomfort in the eye are described as burning, stinging, smarting, itch, and pain with various levels of intensity. Itch is characteristic of conjunctivitis and excruciating pain is typical of corneal injury. Many local anesthetics, such as procaine, cocaine, lidocaine and tetracaine, at concentrations of 0.25 to 4%, can readily suppress eye discomfort, including corneal pain, but any prolonged use of these agents in the eye is associated with significant damage to the corneal epithelium because of inhibition of cell turnover. This toxic effect of local anesthetics greatly limits their use. A new class of safer chemical agents, capable of suppressing eye irritation, itch and pain, is the subject of this invention.

Ophthalmic Drug Delivery:

The most common method of ocular drug delivery is the instillation of drops into the lower eyelid (i.e., “eye drops”). About 70% of prescriptions for eye medication are for eye drops. This is due to factors such as expense, ease of bulk manufacture, and patient compliance, as well as effective and uniform drug delivery. A key requirement is that the formulation be sterile and produced in a sterile environment. An ideal active ingredient in ophthalmic solutions should be soluble and/or miscible in aqueous media at normal ocular pH and tonicity. Moreover, the drug should be stable, non-toxic, long acting, and sufficiently potent to counteract dilution of drug concentration by blinking and tearing.

Compositions of the Present Invention:

Broadly, eye drop compositions in accordance with the present invention include a trialkyl phosphine oxide as an essential component in a pharmaceutically effective amount The general formula for trialkyl phosphine oxides, Formula 1, is represented below:
(R₁R₂R₃)P═O Formula 1

Compounds of highest activity and preferred for use in the present invention are those wherein R₁ is a straight chain alkyl group of from 5-8 carbon atoms, wherein R₂ is a branched chain alkyl group of from 3-5 carbon atoms, (R₂ especially being an isopropyl, secondary-butyl, isobutyl, tertiary-butyl, an isopentyl group or a cycloalkyl group); and wherein R₃ is an alkyl group, preferably a branched chain alkyl group, of from 3-6 carbon atoms, preferably 4 or 5 carbon atoms and R₁, R₂ and R₃ providing a total of from 13-17 carbon atoms. Branching in this context is to be taken to include cyclic structures, as well as branched chain acyclic groups. Table 1 below shows a number of suitable trialkyl phosphine oxides with particularly preferred ones being indicated by an asterisk in column (e).

TABLE 1
Trialkyl Phosphine Oxide Compounds.

| | | | | | c. | | e. |
|---|---|---|---|---|---|---|---|
| | | | a. | b. | Duration | d. | Eye |
| | | | Threshold | Relative | of Action | Sensory | Therapy |
| R₁ | R₂ | R₃ | μg/ml | Potency | (min) | Quality | μg/ml |
| i-C₅H₁₁ | n-C₃H₇ | n-C₃H₇ | >250 | <0.005 | 5 | cool/sting | >1500 |
| n-C₅H₁₁ | n-C₅H₁₁ | n-C₂H₅ | >150 | <0.008 | 5 | cool/sting | >1000 |
| n-C₈H₁₇ | n-C₆H₁₃ | n-C₆H₁₃ | >250 | <0.005 | 10 | cool/sting | >1500 |
| n-C₁₀H₂₁ | iso-C₅H₁₁ | iso-C₅H₁₁ | >250 | <0.005 | 10 | cool/sting | >1500 |
| n-C₉H₁₉ | iso-C₄H₉ | iso-C₄H₉ | 50 | 0.025 | 15 | cool/sting | >250 |
| n-C₆H₁₃ | n-C₆H₁₃ | n-C₂H₅ | 20 | 0.06 | 5 | cool/sting | >150 |
| n-C₇H₁₅ | n-C₇H₁₅ | tert-C₄H₉ | 20 | 0.06 | 10 | cool/sting | >150 |
| iso-C₅H₁₁ | iso-C₅H₁₁ | sec-C₄H₉ | 20 | 0.06 | 5 | cool/sting | >150 |
| n-C₆H₁₃ | tert-C₄H₉ | n-C₃H₇ | 3.5 | 0.36 | 7.5 | cool only | 30* |
| n-C₇H₁₅ | sec-C₄H₉ | sec-C₄H₉ | 1.5 | 0.8 | 10 | cool/sting | 15 |
| n-C₆H₁₃ | tert-C₄H₉ | n-C₄H₉ | 1.5 | 0.8 | 5 | cool only | 25* |
| n-C₆H₁₃ | sec-C₄H₉ | sec-C₄H₉ | 1.25 | 1 | 20 | cool/sting | 45 |
| n-C₅H₁₁ | sec-C₄H₉ | sec-C₄H₉ | 1.25 | 1 | 10 | cool only | 60* |
| n-C₆H₁₃ | iso-C₅H₁₁ | iso-C₃H₇ | 1.25 | 1 | 5 | cool only | 25* |
| n-C₇H₁₅ | iso-C₅H₁₁ | iso-C₃H₇ | 1.25 | 1 | 5 | cool/oily | 20* |
| n-C₈H₁₇ | sec-C₄H₉ | sec-C₄H₉ | 0.5 | 2.5 | 10 | cool/sting | 30 |
| n-C₆H₁₃ | iso-C₃H₇ | sec-C₄H₀ | 0.5 | 2.5 | 45 | cool/sting | 15 |
| n-C₇H₁₅ | iso-C₃H₇ | sec-C₄H₉ | 0.1 | 10 | 18 | cool only | 10* |
| n-C₈H₁₇ | iso-C₃H₇ | sec-C₄H₉ | 0.1 | 10 | 18 | cool only | 10* |

a.) the threshold of the substance for producing cooling sensations when applied to the tongue of human volunteers
b.) the reciprocal value of the threshold is the relative potency, normalized to unity for analogs with a 1.25 μg/ml potency
c.) the duration of the cooling action on the tongue is given in minutes
d.) sensory quality of the solution when applied on the medial canthus of the eyelids
e.) an estimate of the ideal therapeutic concentration of the trialkyl phosphine oxide to be applied as eye drops to relieve eye disorders
*particularly preferred compounds for therapy based on potency, duration of action, and sensory quality

Thus, particularly preferred trialkyl phosphine oxides have the Formula 1 structure wherein R₁ is n-C₅H₁₁, n-C₆H₁₃, n-C₇H₁₅ or n-C₈H₁₇, R₂ is iso-C₃H₇, sec-C₄H₉, tert-C₄H₉ or iso-C₅H₁₁and R₃ is n-C₃H₇, iso-C₃H₇, sec-C₄H₉, or n-C₄H₉.

In the present invention, preferred formulations including trialkyl phosphine oxides are eye drops, a form of drug delivery that is pharmaceutically-acceptable to patients, convenient, safe, with an onset of action of several minutes. A standard eye drop used in therapy according to federal regulatory practice is sterile, is isotonic (i.e., a pH of about 7.4 for patient comfort), and, if to be used more than once, contains a preservative but has a limited shelf life after opening, usually one month. If the eye drops are packaged in a sterile, single use only unit-dose dispenser the preservative may be omitted.

A preferred method of eye drop formulation is to take the purest form of the trialkyl phosphine oxide drug (e.g., greater than 99% purity), and mix this liquid with purified water and adjust for physiological pH and isotonicity. Examples of buffering agents to maintain or adjust pH include, but are not limited to, acetate buffers, citrate buffers, phosphate buffers and borate buffers. Examples of tonicity adjustors are sodium chloride, mannitol and glycerin. As will be further discussed hereinafter, additional components may also be added.

The formulated solution is then aliquoted into either a plurality of discrete, sterile disposable cartridges each of which is suitable for unit dosing, or a single cartridge for unit dosing. Such a single disposable cartridge may be, for example, a conical or cylindrical specific volume dispenser, with a container having side-walls squeezable in a radial direction to a longitudinal axis in order to dispense the container contents therefrom at one end of the container. Such disposable containers are currently used to dispense eye drops at 0.3 to 0.4 ml (e.g., Lens Plus® and Refresh Plus®) per unit dosing, and are ideally adaptable for the delivery of eye drops. W.S. Iba, in U.S. Pat. No. 5,582,330, incorporated herein by reference, describes an example of such a unit volume dispenser for eye drops.

Ophthalmic eye-drop solutions are also packaged in multidose form, for example, as a plastic bottle with an eye-dropper (e.g., Visine® Original). In such formulations, preservatives are required to prevent microbial contamination after opening of the container. Suitable preservatives include, but are not limited to: benzalkonium chloride, thimerosal, chlorobutanol, methyl paraben, propyl paraben, phenylethyl alcohol, edetate disodium, sorbic acid, polyquatemium-1, or other agents known to those skilled in the art, and all of which are contemplated for use in the present invention. Such preservatives are typically employed at a level of from 0.001 to about 1.0% weight/volume.

Eye drops provide a pulse entry of the drug, but the drug is rapidly diluted by tears and flushed out of the eye. Polymers are frequently added to ophthalmic solutions in order to increase the viscosity of the vehicle; this prolongs contact with the cornea, often enhancing bioavailability. The types of polymers permitted by the Federal Food and Drug Administration in ophthalmic solutions are defined concentrations of cellulose derivatives (methylcellulose, hydroxyethylcellulose, hydroxypropylcellulose and carboxymethylcellulose), dextran 70, gelatin, polyols, glycerin, polyethylene glycol 300, polyethylene glycol 400, polysorbate 80, propylene glyclol, polyvinyl alcohol and povidone, all of which (singly or in combination) are contemplated for use in the present invention.

In certain clinical conditions, the trialkyl phosphine oxide eye drop solutions may be formulated with other pharmaceutical agents, in order to attenuate the irritancy of the other ingredient and to facilitate clinical response. Such agents may include, but are not limited to, a vasoconstrictor such as phenylephrine, oxymetazoline, napthazoline or tetrahydrozoline; a mast-cell stabilizer such as olopatadine; an antihistamine such as azelastine; an antibiotic such as tetracycline; a steroidal anti-inflammatory drug such as betamethasone; a non-steroidal anti-inflammatory drug such as diclofenac; an immunomodulator such as imiquimod or interferons; and antiviral agents such as valaciclovir, cidofovir and trifluridine. The doses used for the above described purposes will vary, but will be in an effective amount to suppress discomfort, itch, irritation, or pain in the eye. When the compositions are dosed topically, the “pharmaceutically effective amount” of trialkyl phosphine oxide will generally be in a concentration range of from 0.0001 to about 0.1% weight/volume, with 1 to 4 drops administered as a unit dose 1 to 4 times per day.

I found that there was a general correlation of potencies between cooling effects on the tongue and therapeutic activity on the eye surface (Table 1), but the relationship was not precise. The therapeutic concentrations of the preferred analogs are in the range of 10 to 80 μg/ml (0.01 to 0.08%), but concentrations as low as 0.1 μg/ml may elicit effects in sensitive individuals. In contrast to other water-soluble drugs used as eye drops, such as antiviral agents, a super potent trialkyl phosphine oxide analog does not necessarily confer pharmaceutical advantage because at concentrations below 1 μg/ml possible adsorptive loss of the active ingredient on plastic surfaces, e.g. on unit dispensers, may interfere with drug delivery. Therefore in practicing this invention, therapeutic formulations can also be prepared with less potent analogs in the range of 1 mg to 10 mg/ml (0.1 to 1%).

The potency of certain specific trialkyl phosphine oxides, e.g. isopropyl, sec-butyl, n-octyl-phosphine oxide, in relieving eye discomfort is exceptionally high, on the order of 0.001%. In such situations, an alternative preferred description of an effective unit dose is 0.01 mg/ml or 10 μg/ml, which is equivalent to 0.001% weight percent.

One exemplary benefit or utility of this invention is the treatment of corneal pain. The epithelium (outermost layer) of the cornea is densely innervated by sensory fibers. Therefore, the cornea is very sensitive and any damage to the surface epithelium causes severe and sometimes extreme pain. Such injuries are frequently caused by corneal drying, infection and inflammation (which damage epithelial cells), by corneal dystrophies with loosely adherent epithelium, by mechanical removal of the corneal epithelium in traumatic abrasions or surgical procedures (such as LASIK or PRK), or by accidental penetration of the cornea by foreign objects. In most cases, the pain persists until the damaged epithelium heals, which usually takes several days for surgical trauma and may last much longer if there is corneal ulceration. As mentioned previously, local anesthetics cannot be applied frequently for corneal pain because of toxicity. At present, there is a need for effective drugs for pain caused by injury to the corneal epithelium.

This invention may also have utility in several other ocular conditions for which current therapy is unsatisfactory. For example, severe conjunctivitis can occur in infections by viruses such as herpes simplex and herpes zoster, in atopic eczema patients, and in food allergies. Eye drops containing antihistamines and/or mast-cell stabilizers are relatively ineffective for controlling the symptoms of conjunctivitis in such situations and trialkyl phosphine oxide eye drops may provide a better alternative. The dry eye syndrome (keratoconjunctivitis sicca) is not a life-threatening disorder, however, because of the sheer number of soft contact lens users and an aging population that have this disability, a trialkyl phosphine oxide eye drop drug that promotes visual refreshment (see Example 3) will have value in treatment of this condition.

As earlier noted, most of the trialkyl phosphine oxides produce a degree of stinging and burning sensations when applied directly to the eye when the eyelids are open. These stinging/burning sensations are limiting factors on effective therapy unless the doses are adapted for therapeutic efficacy in treating eye discomfort to reduce or eliminate irritancy that can occur from the trialkyl phosphine oxides. Practice of the invention overcomes this limitation in one or more of the following ways: appropriately choosing the method of delivery, selecting particularly preferred trialkyl phosphine oxide analogs, and the addition of an adjunct to the eye drop to reduce irritancy.

Method of Delivery

The normal standard method of administering ophthalmic solutions is to keep the eyes open. For example, in the instructions for using an eye wash (Bausch and Lomb), the user is advised to fill the cup half with solution, apply the cup tightly to the affected eye, tilt the head backward, open eyelids wide and rotate eyeball thoroughly to wash the eye. For eye drops, Hecht (Chpt 43, Ophthlamic Solutions, pg. 821-835, in “Remington, the Science and Practice of Pharmacy,” Alfonso R. Gennaro, Editor. 20th ed. Baltimore, Md.: Lippincott Williams & Wilkins, 2000) states that the dropper is held above the eye and drops applied to the lower eyelid when the subject is looking upwards. The lower eyelid is then released, the subject is advised to keep the eye open and not blink for at least 30 seconds.

For trialkyl phosphine oxide eye drop solutions of this invention, I have discovered an indirect method of application as follows. The eye dropper is aimed at the medial canthus (nasal corner) of the eye, the eye is then closed, the dispenser is squeezed to emit the eye drops, and the eye kept closed for about 1 min. This method of application can substantially avoid stinging and burning sensations. Apparently, the adaptation period of about 1 min allows the sensory processes to develop tolerance to the initial stinging and burning stimuli. For example, if a 0.4 mg/ml solution of disec-butyl, n-hexyl-phosphine oxide [synonym, 1-(Di-sec-butyl-phosphinoyl)-hexane] in isotonic-buffered saline is dropped on the open eye, it causes stinging and burning for about 1 min. However, applied onto the corners of the eye, with the eyes closed for 1 min, these stinging and burning sensations are avoided. The medial canthus is preferred to the lateral canthus because the geometry of the orbit permits a longer residence time for the eye drop applied to the medial corner. Drops applied to the lateral canthus are less precisely located and rapidly dribble off the zygoma (cheekbone). A user can be instructed in this indirect method by including the instructions on packaging labels and/or inserts associated with the doses.

Another modification of drug delivery was discovered. In this alternative indirect method of drug application, the trialkyl phosphine oxide is applied to the skin above the eyeball but does not touch the margins of the palpebral fissure or eye surface. Example 4 notes that topical application of a 5% solution of a trialkyl phosphine oxide dissolved in pure propylene glycol produces robust cooling when applied in this manner.

Selected Trialkyl Phoshine Oxide

In selecting a particular analog, it is desirable to have one that has a long duration of action (several hours) and minimal stinging and burning sensations. These parameters are not predicted by the cooling effect of these substances on the human tongue, but must be determined by experiment. For example, I found that disec-butyl-n-hexyl phosphine oxide and disecbuytl-n-heptyl phosphine oxide are about equipotent on the human tongue, yet on the eye surface the heptyl analog has half the duration of action and is three times more active in producing stinging and burning sensations. Thus, the hexyl analog is the preferred therapeutic of the two. The other particularly preferred analogs that have the desired qualities of long duration of action and absence of stinging and burning effects are identified with an asterisk in Table 1.

Adjunct to Reduce Irritancy

I found that the stinging and burning sensations of several trialkyl phosphine oxide solutions (see Examples) were substantially reduced in the presence of 1 to 2% sucrose and in the presence of other carbohydrates such as fructose, dextrose (D-glucose), inositol and 0.5% carboxymethylcellulose. The reduction in stinging and burning sensations was not correlated to sweetness because a synthetic chlorinated carbohydrate sweetener, sucralose (600 times more potent than sucrose), was not active in reducing stinging and burning sensations at equivalent sweetening concentrations (see example). I conclude that hydrocarbon polyols are useful adjuncts for preparing a therapeutic formulation of a trialkyl phosphine oxide eye drop solution.

In summary, I describe methods of treating discomfort and pain in the eye by administering droplets of an inventive composition containing a trialkyl phosphine oxide in an ophthalmic solution. The preferred method of administration is to aim and drip the solution onto the nasal corner (medial canthus) of the closed eye and to keep the eye closed until at least one minute after instillation. The preferred trialkyl phosphine oxide is selected for potency, long duration of action, and the absence of irritancy. A hydrocarbon polyol or a similar demulcent may be further added to the composition in order to further reduce irritancy. The concentration of the trialkyl phosphine oxide in the ophthalmic solution is preferably in an amount of at least about 0.0001 wt. % to about 0.5 wt. % of the composition, more preferably in the range of 0.007 to 0.1 wt. %, and most preferably in the range of 0.01 to 0.06 wt. %. The composition may further include a suitable demulcent (0.01 to 4%) or a hydrocarbon polyol in an amount of about 0.5 to 3%, or a carbohydrate monomer at a molar ratio of monomer: trialkyl phosphine oxide of 10:1 to 50:1).

### EXPERIMENTAL

### EXAMPLE A, PREPARATION OF DISEC-BUTYL, N-HEXYL-PHOSPHINE OXIDE

Preparations of trialkyl phosphine oxides are disclosed by Rowsell and Spring in U.S. Pat. No. 4,070,496, issued Jan. 24, 1978, herein incoporated by reference. If two alkyl groups are the same, then dialkylphosphinyl chlorides, (R₁R₁)POCl, may be prepared by the action of Grignard reagents on phosphite, after chlorination. For example, disec-butyl-phosphinite is prepared from the addition of sec-butyl-magnesium bromide to diethyl-phosphite (these reagents are available commercially, e.g. from Sigma-Aldrich Chemical Co.). The resulting product is chlorinated and the dialkyl phosphinyl chloride can then be further reacted with another specified Grignard reagent to form the desired trialkyl phosphine oxides. If all three alkyl groups of a tertiary phosphine oxide are different, then the alternative reaction between a Grignard reagent, RMgX, and chlorophosphite gives dialkyl phosphinite. This latter compound, when allowed to react with another Grignard reagent, R′MgX, will give the unsymmetrical secondary phosphine oxide, (RR′)(OH)P═O. Trialkyl phosphine oxides can be prepared from these secondary phosphine oxides. Some of these standard reactions are described in Organic Phosphorus Compounds, Volumes 3 and 4, edited by G. M. Kosolapoff and L. Maier, published by Wiley-Interscience, 1972. The general principles for the reaction of Grignard reagents with halides and esters of phosphorous acids are well-known to practitioners of the art (G. M. Kosolapoff. Organophosphorus Compounds, New York, Wiley, pg. 107 to 109, 1950).

For example, in the preparation of disec-butyl-n-hextylphosphine oxide, a solution of disec-butylphosphinyl chloride (3.9 gm) in tetrahydrofuran (50 ml) was added dropwise to a refluxing solution of n-hextylmagnesium bromide. The mixture was heated under reflux for 18 hours. After cooling to room temperature, the reaction mixture was poured onto ice and 2N HCl (300 ml), and extracted with methylene dichloride. The combined extracts were washed with lithium hypochlorite solution, 2N NaOH solution and finally with water, then dried (with MgSO₄). The solvent was removed by distillation and the residual yellow oil (8 gm) was eluted with chloroform down a silica gel column. The product (R_{f}=0.1 to 0.2 on silica thin layer chromatography (CHCl₃)) was finally distilled to yield disec-butyl-n-hextyl-phosphine oxide as a colorless liquid, boiling point 120° C. This compound was then subjected to bioassays.

### EXAMPLE 1

A 0.05% (0.5 mg/ml) disec-butyl-n-hexyl-phosphine oxide eye drop solution was prepared by adding the compound to an isotonic solution of sodium chloride, 0.65% in deionized water, monobasic potassium phosphate/sodium hydroxide buffer, preserved with disodium EDTA and benzalkonium chloride. The liquid was individually aliquoted into a ¼ oz. bottle with a Yorker spout (E.D. Luce Packaging) suitable for droplet delivery. This solution was applied to the opened eyes of three volunteers, with two to three drops of the solution applied to each eye. The subjects complained of stinging and burning sensations on the eye surface, lasting for about 2 min and requiring the subjects to shut their eyes. Afterwards, the stinging sensations disappeared and were replaced by cooling sensations on the eyelids and eye surfaces lasting for about 1.5 to 2 hours. There was no evidence of increased redness or other indications of vasodilatation in the blood vessels on the eye surface or eyelids, nor subsequent complaints of discomfort.

### EXAMPLE 2

The solution, as in Example 1, was then used by another three subjects with the instruction to apply the droplets to the nasal corner of the eye (medial canthus), without touching the tip of the dispenser to the eyelid surface, to let the solution seep into the eyelids and eye surface but to keep eyes closed for at least one minute. The subjects reported slight sensations of stinging when the eyes were opened, effects that lasted for less than 1 minute, followed by sensations of coolness and comfort lasting at least two hours. The experiment was then repeated, but with a higher concentration of 0.2% of disec-butyl-n-hexyl-phosphine oxide. Sensations of stinging and burning were slightly increased, but the cooling and refreshing actions were much stronger, lasting 3 to 5 hours. The subjects reported that the eyes felt “wet and cool”, but no signs of tearing was observed, nor of signs of irritation or redness.

### EXAMPLE 3

A 0.2% (2.0 mg/ml) disec-butyl-n-heptyl-phosphine oxide eye drop solution was prepared by adding the compound to an isotonic solution of sodium chloride, 0.65% in deionized water, monobasic potassium phosphate/sodium hydroxide buffer, preserved with disodium EDTA and benzalkonium chloride. The liquid was individually aliquoted into a ¼ oz. bottle with a Yorker spout (E.D. Luce Packaging) suitable for droplet delivery. This solution was then applied to the closed eyes of three volunteers, with two to three drops of the solution applied to each eye. The subjects complained of stinging and burning sensations on the eye surface, lasting for about 2 min. A series of six solutions were then prepared in which six different hydrocarbon polyols were added to the parent 0.2% solution. The individual additions were 1.5% sucrose, 1.5% fructose, 1.5% dextrose, 1.5% lactose, 1.5% inositol, or 0.5% carboxymethylcellulose. These adjuncts were found to reduce the initial stinging sensations produced by the 0.2% trialkyl phosphine oxide solutions. There was some tearing, but no evidence of increased eye redness from the hydrocarbon polyols and subjective complaints of discomfort were minimal.

### EXAMPLE 4

A female subject, aged 35, was stuck in traffic for over 2 hours on a hot and sunny (95° to 97° F.) day in an old car without air-conditioning. She was a user of soft contact lenses. Upon returning home, her eyes felt irritated and red, and she had a headache. She wanted to go on-line to work on a database project, but her eyestrain hindered her ability to focus. She volunteered to apply onto the skin of her upper eyes a 5% solution of disec-butyl-n-hexyl-phosphine oxide dissolved in 100% propylene glycol. A cotton swabstick was used to dab the solution onto the surfaces of the skin with extra care not touch the margins of the eyelids or eye surface. After drug application, a robust cooling sensation developed in the eyes that lasted for about 3 hours. The individual remarked that she could feel the coolness through the skin of the upper eyelids and that the sensations were pleasant, refreshing and alleviated her sense of eye fatigue, irritation, and discomfort. She also reported that she felt more alert and energetic.

### EXAMPLE 5

The female subject of example 4 is a daily user of disposable soft contact lenses, has “dry eyes”, and is a constant user of Refresh Eyes®, an artificial tears solution with 0.5% carboxymethylcellulose stored in a unit dispenser. She was instructed on how to instill the eye drops into the nasal corner (medial canthus) of her closed eyes and how to avoid the initial discomforts of the solution. The subject, who is an internationally-ranked tennis player, volunteered to self-administer, by the medial canthus/closed eye method, a 0.2% solution disec-butyl-n-hexyl-phosphine oxide ophthalmic solution containing 0.5% carboxymethylcellulose. She reported a slight tingling in her eyelids followed by refreshing sensations in her eyelids and eye surface, better visual acuity, and a sense of heightened visual awareness. Her eyes felt wet but there was no increased tearing. She was of the opinion that the eye drops were useful to alleviate eye dryness and that tennis players and other athletes may benefit from use of these eye drops in competitions.

### EXAMPLE 6

A male subject, aged 58, suffered from seasonal allergy. During the months of March through May, he would wake up in the morning sneezing, with a runny nose, and itchy eyes. The rhinitis, nasal congestion and obstruction were relieved to some extent by taking a Claritin® antihistamine tablet, but the symptoms of itchy, tearing, and red eyes were not. This individual applied, by the medial canthus/closed eye method in which he had been instructed, several drops of a 0.1% solution disec-butyl-n-pentyl-phosphine oxide ophthalmic eye drop solution, into each eye about every 8 hours for two days. He reported an initial mild sensation of “stinging”; however, the itching was then substantially reduced, and the redness was diminished, but there was still some tearing. He felt more clear-sighted and vigilant, and was of the opinion that the eye drops had therapeutic value for his conjunctivitis. Similar results were obtained with 3 additional subjects suffering from ocular allergies.

### EXAMPLE 7

A male subject, aged 87, suffered from perennial rhinitis that was aggravated by sinusitis, nasal surgery, and a prolonged course of antibiotics. He developed a generalized allergic condition with severe itchy rash on his back, dermatitis on his face (especially on his cheeks), and had itchy, puffy and red eyes. This individual applied, by the medial canthus/closed eye method in which he had been instructed, several drops of a 0.1% solution disec-butyl-pentyl-phosphine oxide ophthalmic solution into each eye for about every 8 hours for three days. No significant stinging sensations were noted. He remarked that the solution produced a sense of eye “wetness” and relief of discomfort. The subject was of the opinion that the eye drops had therapeutic value for his conjunctivitis.

### EXAMPLE 8

A male subject, aged 37, underwent bilateral refractive eye surgery (LASIK) for myopia in order to maintain his qualifications for special duties in the military. On the first day after surgery his eye surfaces and eyelids were bright red from dilated and enlarged blood vessels, but there was little pain. However, on the second day, the individual suffered excruciating pain from his eyes that was incapacitating and prevented him from going to work. This individual volunteered to apply eye drops of a 0.05% solution disec-butyl-n-hexyl-phosphine oxide ophthalamic into each eye for about every 6 hours for three days. He was instructed on how to instill the eye drops into the nasal corner (medial canthus) of his closed eyes and how to avoid the initial discomforts of the solution. He reported that the acute pain from the eye surface was significantly diminished within minutes after instillation of the eye drops, but that there was still some tearing. He felt much better and was of the opinion that the eye drops had therapeutic value for ocular pain. This individual's eye function and appearance returned to normal within 7 days and no significant adverse effects were observed.

It is to be understood that while the invention has been described above in conjunction with preferred specific embodiments, the description and examples are intended to illustrate and not limit the scope of the invention, which is defined by the scope of the appended claims

1. An eye drop composition, useful to reduce eye discomfort, comprising: one or more doses of a buffered, isotonic ophthalmic solution having therein a pharmaceutically effective amount of a trialkyl phosphine oxide of Formula 1
(R₁R₂R₃)P═O Formula 1
wherein R₁ is an alkyl radical containing at least 3 carbon atoms, R₂ is an alkyl radical containing at least 3 carbon atoms or a cycloalkyl radical, R₃ is an alkyl radical, and R₁, R₂ and R₃ total of from 13-17 carbon atoms, wherein the one or more doses are adapted for therapeutic efficacy in treating eye discomfort by including one or more of:
a.) a selection of R₁ as n-C₅H₁₁, n-C₆H₁₃, n-C₇H₁₅ or n-C₈H₁₇, R₂ as iso-C₃H₇, sec-C₄H₉, tert-C₄H₉ or iso-C₅H₁₁ and R₃ as n-C₃H₇, iso-C₃H₇, sec-C₄H₉, or n-C₄H₉;
b.) an adjunct to reduce irritancy from the trialkyl phosphine oxide; and
c.) instructions to the user for applying the solution indirectly to the eye.

2. The eye drop composition as in claim 1 wherein the eye drop composition is substantially non-astringent.

3. The eye drop composition as in claim 1 wherein the adjunct, if present, is an ophthahnic demulcent.

4. The eye drop composition as in claim 1 wherein the adjunct, if present, is a hydrocarbon polyol.

5. The eye drop composition as in claim 1 wherein the instructions, if present, are carried on packaging associated with the one or more doses.

6. The eye drop composition as in claim 1 wherein the instructions, if present, are on an insert associated with the one or more doses.

7. The eye drop composition as in claim 1 wherein the trialkyl phosphine oxide is in a concentration of from about 0.001 weight percent to about 0.5 weight percent (10 μg/ml to 5 mg/ml) per dose.

8. A method of reducing eye discomfort in a user, comprising: providing a buffered, isotonic ophthalmic solution having therein a pharmaceutically effective amount of a trialkyl phosphine oxide of Formula I
(R₁R₂R₃)P═O Formula 1
wherein R₁ is an alkyl radical containing at least 3 carbon atoms, R₂ is an alkyl radical containing at least 3 carbon atoms or a cycloalkyl radical, R₃ is an alkyl radical, and R₁, R₂ and R₃ total of from 13-17 carbon atoms, wherein the solution is either provided as a unit dose or is determinable as a unit dose; and, instructing the solution user to administer the unit dose onto the nasal corner (medial canthus) of an eye and to keep the eye closed for at least one minute after the administration.

9. The method as in claim 8 wherein the dose is administered to the nasal corner while the eye is closed.

10. The method as in claim 8 wherein the trialkyl phosphine oxide is in a concentration of from about 0.001 weight percent to about 0.5 weight percent (10 μg/ml to 5 mg/ml) per dose.

11. The method as in claim 8 wherein the solution includes an adjunct to reduce irritancy from the trialkyl phosphine oxide.

12. The method as in claim 11 wherein the adjunct is an ophthalmic demulcent.

13. The method as in claim 11 wherein the adjunct is a hydrocarbon polyol.

14. An ophthalmic composition, comprising:
a pharmaceutically effective amount of a trialkyl phosphine oxide of Formula 1
(R₁R₂R₃)P═O Formula 1
wherein R₁ is R₁ is n-C₅H₁₁, n-C₆H₁₃,n-C₇H₁₅or n-C₈H₁₇, R₂ is iso-C₃H₇, sec-C₄H₉, tert-C₄H₉ or iso-C₅H₁₁ and R₃ is n-C₃H₇, iso-C₃H₇, sec-C₄H₉, or n-C₄H₉.

15. The composition as in claim 14 wherein the trialkyl phosphine oxide is carried in a buffered, isotonic solution.

16. The composition as in claim 15 wherein the solution is substantially non-astringent.

17. The composition as in claim 15 wherein the trialkyl phosphine oxide is in a concentration of from about 0.001 weight percent to about 0.5 weight percent (10 μg/ml to 5 mg/ml) per dose

18. A method of relieving eye discomfort comprising:
administering a dose of an ophthalmic composition, comprising:
a trialkyl phosphine oxide of Formula 1
(R₁R₂R₃)P═O Formula 1
wherein R₁ is n-C₅H₁₁, n-C₆H₁₃, n-C₇H₁₅ or n-C₈H₁₇, R₂ is iso-C₃H₇, sec-C₄H₉, tert-C₄H₉ or iso-C₅H₁₁ and R₃ is n-C₃H₇, iso-C₃H₇, sec-C₄H₉, or n-C₄H₉, trialkyl phosphine oxide administered being in a pharmaceutically effective amount.

19. The method as in claim 18 wherein wherein the trialkyl phosphine oxide is in a concentration of from about 0.001 weight percent to about 0.5 weight percent (10 μg/ml to 5 mg/ml) per dose.

20. The method as in claim 18 wherein the ophthalmic composition is substantially non-astringent.
